(19) United States
(12) Patent Application Publication
(10) Pub. No.: US 2022/0202057 A1
(43) Pub. Date: Jun. 30, 2022

(54) METHOD FOR CONTROLLING FOOD PRINTER

(71) Applicant: Panasonic Intellectual Property Management Co., Ltd., Osaka (JP)

(72) Inventors: HIROSHI YAHATA, Osaka (JP); TAKAHIRO NISHI, Nara (JP); TADAMASA TOMA, Osaka (JP); TOSHIYASU SUGIO, Osaka (JP); CHRISTOPHER JOHN WRIGHT, London (GB); BERNADETTE ELLIOTT BOWMAN, London (GB); DAVID MICHAEL DUFFY, Zurich (CH)

(21) Appl. No.: 17/695,361

(22) Filed: Mar. 15, 2022

Related U.S. Application Data
(63) Continuation of application No. PCT/JP2021/014672, filed on Apr. 6, 2021.

(30) Foreign Application Priority Data
Apr. 6, 2020 (JP) 2020-068602
Apr. 6, 2020 (JP) 2020-068603

(51) Int. Cl. A23P 30/00; A23P 20/25

(52) U.S. Cl. CPC A23P 30/00; A23P 2020/253; A23P 20/25

(57) ABSTRACT

A method includes: acquiring chewing/swallowing information from a sensing device installed on a user, wherein the chewing/swallowing information is related to chewing of the user when the user eats a first printed food; determining based on the chewing/swallowing information, a number of chews made by the user, and determining based on a first print pattern and the number of chews, a second print pattern for a second printed food to be created by a food printer; and transmitting print control information to the food printer via a network, wherein the print control information is used for causing the food printer to create the second printed food using the determined second print pattern.

### BACKGROUND

### 1. Technical Field

The present disclosure relates to a method for controlling a food printer.

### 2. Description of the Related Art

Japanese Unexamined Patent Application Publication No. 2014-054269 discloses an oral function training implement that makes it possible to recover, maintain, or improve oral function, and allows training to be performed in a manner similar to the actual swallowing motion. Specifically, the oral function training implement disclosed in Japanese Unexamined Patent Application Publication No. 2014-054269 includes a grip, and an insertion unit designed for insertion into the oral cavity. The insertion unit is provided with a flexible elastic body with a hollow area defined therein. The elastic body includes a hole, and a slit that communicates the hollow area with the outside.

International Publication No. 2014/190168 discloses a 3D printer used for food manufacture.

### SUMMARY

One non-limiting and exemplary embodiment provides further improvements over the techniques described in Japanese Unexamined Patent Application Publication No. 2014-054269 and International Publication No. 2014/190168.

In one general aspect, the techniques disclosed here feature a method for controlling a food printer in a food-material providing system. The food printer is a food printer that creates a first printed food. The first printed food is created by the food printer by using a first print pattern. The method includes: acquiring chewing/swallowing information via a network from a sensing device associated with a user, wherein the chewing/swallowing information is related to chewing of the user when the user eats the first printed food; determining based on the chewing/swallowing information, a number of chews made by the user in eating the first printed food, and determining based on at least the first print pattern and the number of chews, a second print pattern used for a second printed food to be created by the food printer; and transmitting print control information to the food printer via the network, wherein the print control information is used for causing the food printer to create the second printed food using the determined second print pattern.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram illustrating an exemplary general configuration of an information system according to an embodiment of the present disclosure;

FIG. 2 illustrates an exemplary data structure of a chewing/swallowing information database;

FIG. 3 is a sequence diagram illustrating an overview of processing performed by the information system illustrated in FIG. 1;

FIG. 4 is a flowchart according to the embodiment, providing a detailed illustration of processing performed by a server; and

FIG. 5 illustrates the progression of the mean number of chews over time.

### DETAILED DESCRIPTIONS

### Underlying Knowledge Forming Basis of the Present Disclosure

Chewing function and swallowing function (to be referred to as “chewing and swallowing function” hereinafter) are known to decrease with aging. Severe impairment of chewing and swallowing function may have consequences such as deteriorated nutritional status resulting from the inability or difficulty to eat and drink, decreased quality of life (QOL) resulting from the loss of the pleasure of eating, and development of aspiration pneumonia resulting from entry of food or drink into the airway. Aspiration pneumonia, in particular, is among the leading causes of death for the elderly. Accordingly, it is becoming an urgent issue to improve the chewing and swallowing function of the elderly.

If a soft food is provided to an elderly person with decreased chewing and swallowing function for the reason that such a food is easy to eat, this may temporarily allow the elderly person to smoothly ingest the food. However, continuing to provide such a food to the elderly person may further exacerbate the deterioration of the chewing and swallowing function of the elderly person.

Conversely, if a food that requires much chewing is given to an elderly person, it takes a greater number of chews, a longer swallow cycle duration, and a longer meal duration for the elderly person to eat the food. This may make it temporarily impossible or difficult for the elderly person to smoothly ingest the food. However, continuing to provide such a food to the elderly person can potentially improve the chewing and swallowing function of the elderly person. This leads to reduced number of chews for the same food, which results in reduced swallow cycle duration and reduced meal duration.

According to Japanese Unexamined Patent Application Publication No. 2014-054269 mentioned above, the training implement is inserted into the user's oral cavity, and training is performed in a manner similar to the actual swallowing motion. The technique according to Japanese Unexamined Patent Application Publication No. 2014-054269, however, merely involves making the user perform a swallowing motion in a simulated fashion, and does not involve making the user actually chew a real food and perform the actual swallowing motion.

International Publication No. 2014/190168 neither describes nor suggests using food manufactured by a 3D printer to improve the chewing and swallowing function of the elderly.

The above-mentioned knowledge has led the present inventors to discover a method for controlling a food printer that makes it possible to improve the chewing and swallowing function of the user through provision of a food having a suitable size (small bite size), hardness (chewiness), or taste (plain taste).

According to an aspect of the present disclosure, there is provided a method for controlling a food printer in a food-material providing system. The food printer is a food printer that creates a first printed food. The first printed food is created by the food printer by using a first print pattern. The method includes: acquiring chewing/swallowing information via a network from a sensing device associated with a user, wherein the chewing/swallowing information is related to chewing of the user when the user eats the first printed food; determining based on the chewing/swallowing information, a number of chews made by the user in eating the first printed food, and determining based on at least the first print pattern and the number of chews, a second print pattern used for a second printed food to be created by the food printer; and transmitting print control information to the food printer via the network, wherein the print control information is used for causing the food printer to create the second printed food using the determined second print pattern.

According to another aspect of the present disclosure, there is provided a method for controlling a food printer in a food-material providing system. The food printer is a food printer that creates a first printed food. The first printed food is created by the food printer by using a first print pattern. The method includes: acquiring chewing/swallowing information via a network from a sensing device associated with a user, wherein the chewing/swallowing information represents a number of chews made by the user in eating the first printed food; determining based on at least the first print pattern and the number of chews, a second print pattern used for a second printed food to be created by the food printer; and transmitting print control information to the food printer via the network, wherein the print control information is used for causing the food printer to create the second printed food using the determined second print pattern.

According to the above-mentioned configurations, the chewing/swallowing information related to chewing of the user when the user eats the first printed food that uses the first print pattern is acquired from the sensing device via the network. The number of chews made by the user is determined or acquired based on the chewing/swallowing information. The second print pattern is determined based on the determined or acquired number of chews and on the first print pattern. The print control information for causing the food printer to create the second printed food using the determined second print pattern is transmitted to the food printer via the network.

Consequently, based on the number of chews when the user eats the first printed food that uses the first print pattern, a suitable second print pattern for improving the chewing and swallowing function of the user can be determined. This makes it possible to make the food printer create the second printed food using the determined second print pattern, and have the created second printed food eaten by the user. This results in increased number of chews made by the user, which allows for improved chewing and swallowing function of the user.

In the method, the number of chews may include a total number of times chewing is made by the user in eating the first printed food.

According to the above-mentioned configuration, the number of chews can be clearly defined as the number of chews taken for the user to eat the first printed food.

In the method mentioned above, the print control information may include a print condition for, if the number of chews made by the user is less than a predetermined number of chews, creating the second printed food that has a smaller mass per unit volume than the first printed food.

According to the above-mentioned configuration, if the number of chews made by the user is small, the second printed food with a lower density than the first printed food is created. It is known that when a human being takes a meal, the number of chews for the entire meal can be increased significantly without much conscious effort on the user's part by decreasing the amount of food taken per bite. The lower density of the second printed food relative to the first printed food allows for reduced amount of food taken per bite. As a result, provided that the same amount of material is used to create the first printed food and the second printed food, the number of chews taken to eat the second printed food is expected to increase, which can potentially lead to improved chewing and swallowing function of the user.

In the method mentioned above, if the first printed food includes a plurality of chunks of food in a bite size of less than or equal to 15 cubic centimeters, and the number of chews made by the user is less than a predetermined number of chews, the print control information includes a print condition for creating the second printed food that includes a plurality of chunks of food in a bite size of less than or equal to 15 cubic centimeters and that has a mean volume that is less than a mean volume of the plurality of chunks of food in the bite size included in the first printed food.

As described above, it is known from plural experiments that when a human being takes a meal, the number of chews for the entire meal can be increased significantly by decreasing the bite size. Accordingly, if the first printed food is made up of plural bite-sized chunks, and the user's number of chews is less than a predetermined number of chews, the second printed food made up of even smaller bite-sized chunks can be created to increase the number of chews made by the user during a meal. This can potentially lead to improved chewing and swallowing function of the user.

The reason for using a bite size of less than or equal to 15 cubic centimeters is to clearly define what a bite size is. There exist published experimental results indicating that adult males have a mean palatal volume of 12,254 cubic millimeters, and adult females have a mean palatal volume of 10,017 cubic millimeters. From such results, even with differences between individuals or races taken into account, the size of 15 cubic centimeters (15,000 cubic millimeters) is considered to be large enough as a volume representing the size of food eaten by a human being in one bite. Therefore, to provide an objective index of bite size, a bite size of food is defined as a volume of less than or equal to 15 cubic centimeters. Of course, this is only one form of expression used to define a bite size, and there is no problem with using a definition other than 15 cubic centimeters as long as such a definition can be interpreted as representing a bite size from a commonsense viewpoint.

The first printed food is assumed to include plural chunks of bite-sized food. Each bite-sized chunk of food included in the first or second printed food may be created individually, or plural bite-sized chunks of food may be created such that these chunks of food are connected by thin (edible) lines even through their boundaries are clearly defined. Even if plural bite-sized chunks of food are connected by thin edible lines, as long as it is expected that when the user eats these chunks of food, the user will eat each bite-sized chunk portion in one bite, then this will not have any significant impact on the number of chews made by the user.

In the method mentioned above, the print control information may include a print condition for, if the number of chews made by the user is less than a predetermined number of chews, creating the second printed food that has a greater volume of a hard portion than the first printed food. The hard portion has a hardness greater than or equal to a predetermined hardness.

The number of chews made by a human being is known to increase if the food eaten contains a food material that requires much chewing. For example, cutting root vegetables into somewhat large pieces leads to a greater number of chews than cutting root vegetables into small pieces.

According to the above-mentioned configuration, the second printed food can be made to include a larger volume of a portion with a predetermined hardness (predetermined chewiness) than the first printed food. As a result, if the number of chews does not sufficiently increase for a user who has eaten the first printed food, the number of chews can be increased by making the user eat the second printed food. This can potentially lead to improved chewing and swallowing function.

In the method mentioned above, the sensing device may include an acceleration sensor, and the chewing/swallowing information may include acceleration information that represents an acceleration detected by the acceleration sensor.

According to the above-mentioned configuration, the number of chews made by the user is determined based on the acceleration information detected by the acceleration sensor. This makes it possible to accurately determine the number of chews.

In the method mentioned above, the sensing device may be a distance sensor, and the chewing/swallowing information may include distance information that is detected by the distance sensor and that represents a distance to a skin.

According to the above-mentioned configuration, the number of chews is determined based on the distance information detected by the distance sensor and representing the distance to the skin. This makes it possible to accurately determine the number of chews. It is known that when a human being makes a chewing motion, for example, the skin near the ears moves with the chewing motion. Therefore, by observing the movement of the skin in such an area, the number of chews can be acquired.

In the method mentioned above, the sensing device may detect an electromyographic potential, and the number of chews may be determined based on the detected electromyographic potential.

According to the above-mentioned configuration, a sensor that detects an electromyographic potential is used to detect the user's electromyographic potential, and the number of chews made by the user is determined based on the electromyographic potential. This makes it possible to accurately determine the number of chews.

In the method mentioned above, the sensing device may detect chewing sound, and the number of chews may be determined based on the detected chewing sound.

According to the above-mentioned configuration, the number of chews made by the user is determined based on the chewing sound. This makes it possible to accurately determine the number of chews.

In the method mentioned above, the sensing device may include a camera, and the number of chews made by the user may be determined based on a result of image recognition performed by using an image obtained with the camera.

Since the sensing device is implemented as a camera, by applying an image recognition process to an image obtained with the camera, the number of chews can be determined. This can be accomplished by capturing an image of the user during a meal by using a camera installed on a smartphone. The smartphone is installed with an application that determines the number of chews made by the user during meal intake through image recognition. As the user starts the application, and takes a meal while capturing the user's own image, the number of chews can be measured or recorded.

In the method mentioned above, the sensing device may be installed on an autonomous device that performs sensing on the user.

As described above, the sensing device is installed on an autonomous device (robot) that performs sensing on the user. Accordingly, when the user starts eating a meal, the autonomous device is able to move closer to the user, and sense the user's eating condition during the meal to thereby measure information such as what the user is eating and the number of chews. By performing multimodal sensing using plural sensors such as a camera and a microphone, the number of chews can be measured accurately and autonomously.

In the method mentioned above, the sensing device may be installed on eyeglasses of the user.

According to the above-mentioned configuration, the user simply puts on eyeglasses to allow determination of the number of chews made by the user. This makes it possible to determine the number of chews in everyday life of the user.

In the method mentioned above, the sensing device may be installed on a device to be worn around a neck of the user.

According to the above-mentioned configuration, the user simply puts on a neck-worn device (e.g., a necklace or a neck speaker) to allow determination of the number of chews made by the user. This makes it possible to determine the number of chews in everyday life of the user.

In the method mentioned above, the sensing device may be installed on a device to be worn on an ear of the user.

According to the above-mentioned configuration, the user simply puts on an ear-worn device (e.g., an earphone, a headphone, or pierced earrings) to allow determination of the number of chews made by the user. This makes it possible to determine the number of chews in everyday life of the user.

In the method mentioned above, the second printed food may be created by using a plurality of paste materials, and the second print pattern may specify where each of the plurality of paste materials is to be used.

According to the above-mentioned configuration, the food printer prints the second printed food while switching between different paste materials. This makes it possible to provide, for example, different colors, textures, or tastes to different portions of the second printed food.

In the method mentioned above, the second printed food may comprise a three-dimensional structure including a plurality of layers, the plurality of layers including a first layer and a second layer, and the print control information may include a print condition for causing a paste material used for the first layer to be varied from a paste material used for the second layer.

According to the above-mentioned configuration, the second printed food includes plural layers including a first layer and a second layer, and the color, texture, or taste of the first layer can be varied from the color, texture, or taste of the second layer. Consequently, for example, a second printed food with a hard surface (first layer) and a soft interior (second layer) can be created as well. This makes it possible to create a second printed food having a texture such that as the user crushes its hard surface with the teeth, its contents with taste mix with saliva and melt out from the inside. This induces saliva production, which helps to efficiently improve the chewing and swallowing function of the user.

In the method mentioned above, the second printed food may comprise a three-dimensional structure including a plurality of layers, the plurality of layers including a first layer and a second layer, and the print control information may include a print condition for causing a third print pattern used for the first layer to be varied from a fourth print pattern used for the second layer.

According to the above-mentioned configuration, the second printed food includes plural layers including a first layer and a second layer, and the texture or texture sensation of the first layer can be varied from the texture or texture sensation of the second layer. Consequently, for example, a second printed food with a hard surface (first layer) and a soft interior (second layer) can be created as well. This makes it possible to create a second printed food having a texture such that as the user crushes its hard surface with the teeth, its contents with taste mix with saliva and melt out from the inside. This induces saliva production, which helps to efficiently improve the chewing and swallowing function of the user.

In the method mentioned above, the print control information may specify a temperature at which to bake the second printed food.

According to the above-mentioned configuration, the print control information includes information specifying the temperature at which to bake the second printed food. Accordingly, for example, the hardness of the second printed food can be adjusted by controlling or specifying at what temperature each individual portion of the second printed food is to heated with a laser output unit in creating the second printed food, or by controlling or specifying at what temperature and for how long the entire second printed food is to be heated with another food preparation appliance (e.g., an oven) after the second printed food is created.

The present disclosure can be implemented also as a program for causing a computer to execute various characteristic features included in the control method mentioned above, or as a food-material providing system that operates in accordance with the program. It is needless to mention that such a computer program can be distributed via a computer-readable non-transitory recording medium such as a CD-ROM, or via a communications network such as the Internet.

Embodiments described below each represent one specific implementation of the present disclosure. Specific details set forth in the following description of embodiments, such as numeric values, shapes, components, steps, and the order of steps, are for illustrative purposes only and not intended to limit the scope of the present disclosure. Those components in the following description of embodiments which are not cited in the independent claim representing the most generic concept of the present disclosure will be described as optional components. For all embodiments of the present disclosure below, the features of individual embodiments may be used in combination.

### Embodiments

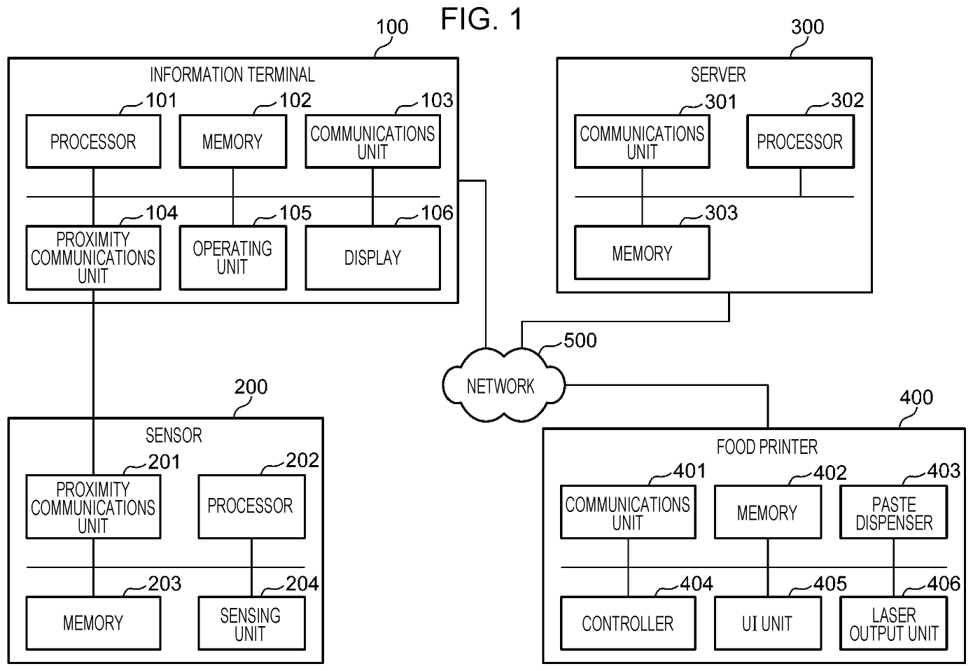

FIG. 1 is a block diagram illustrating an exemplary general configuration of an information system according to an embodiment of the present disclosure. The information system includes an information terminal 100, a sensor 200, a server 300, and a food printer 400. The server 300 and the food printer 400 each represent an example of a food-material providing system. The information terminal 100, the server 300, and the food printer 400 are capable of communicating with each other via a network 500. The information terminal 100 and the sensor 200 are capable of communicating with each other through proximity wireless communication. The network 500 is implemented as, for example, a wide area network including an Internet communications network and a mobile phone communications network. For proximity wireless communication, for example, a wireless technology such as Bluetooth (registered trademark) or NFC is used.

The information terminal 100 is implemented as, for example, a mobile information processing apparatus such as a smartphone or a tablet terminal. However, this is intended to be illustrative only. Alternatively, the information terminal 100 may be implemented as a desktop information processing apparatus.

The information terminal 100 is carried by a user who receives a food-material providing service provided by the food-material providing system. The information terminal 100 includes a processor 101, a memory 102, a communications unit 103, a proximity communications unit 104, an operating unit 105, and a display 106.

The processor 101 is implemented as, for example, a CPU. The processor 101 is responsible for overall control of the information terminal 100. The processor 101 executes the operating system of the information terminal 100, and executes a sensing application for receiving sensing data from the sensor 200 and transmitting the sensing data to the server 300.

The memory 102 is implemented as, for example, a rewritable non-volatile storage device such as a flash memory. The memory 102 stores, for example, the operating system and the sensing application. The communications unit 103 is implemented as a communications circuit for connecting the information terminal 100 to the network 500. The communications unit 103 transmits sensing data to the server 300 via the network 500. The sensing data in this case is sensing data transmitted from the sensor 200 via proximity wireless communication and received by the proximity communications unit 104. The proximity communications unit 104 is implemented as a communications circuit that complies with a proximity wireless communications standard. The proximity communications unit 104 receives sensing data transmitted from the sensor 200.

The operating unit 105 is implemented as an input device such as a touchscreen if the information terminal 100 is implemented as a mobile information processing apparatus. The operating unit 105 is implemented as an input device such as a keyboard and a mouse if the information terminal 100 is implemented as a desktop information processing apparatus. The display 106 is implemented as a display device such as an organic EL display or a liquid crystal display.

The sensor 200 is implemented as a sensing device installed on the user. The sensor 200 includes a proximity communications unit 201, a processor 202, a memory 203, and a sensing unit 204. The proximity communications unit 201 is implemented as a communications circuit that complies with a proximity wireless communications standard. The proximity communications unit 201 transmits sensing data detected by the sensing unit 204 to the information terminal 100.

The processor 202 is implemented as, for example, a CPU, and is responsible for overall control of the sensor 200. The memory 203 is implemented as, for example, a non-volatile rewritable storage device such as a flash memory. The memory 203 temporarily stores, for example, sensing data detected by the sensing unit 204. The sensing unit 204 detects sensing data including information related to user's chewing and/or swallowing (to be referred to as “chewing/swallowing information” hereinafter).

The sensing unit 204 is implemented as, for example, an acceleration sensor. In this case, the acceleration sensor is installed on an eating utensil that the user grips when taking a meal, or to a wearable device installed on the head or upper arm. Exemplary eating utensils include chopsticks, forks, and spoons. Exemplary devices installed on the head or upper arm include wrist-worn smart watches, finger-worn smart rings, smart eyeglasses, ear-worn earphones or sensor devices, and tooth-embedded sensor devices. When the user chews a food, the user raises an eating utensil from a plate to pick up the food on the plate and delivers the food to the mouth, and after placing the picked up food in the mouth, the user lowers the eating utensil toward the plate again. Such motions are repeated during meal intake. Of course, chewing is accompanied by repeated up and down movements mainly around the jaw area. As described above, raising and lowering of an eating utensil or hand, and jaw movements occur in conjunction with the user's chewing motion. Accordingly, acceleration information representative of an acceleration of the eating utensil, an acceleration of the head where chewing-associated movements occur, or an acceleration of the upper arm represents the characteristics of the user's chewing. Accordingly, the embodiment uses, as chewing/swallowing information, acceleration information representative of an acceleration detected by an acceleration sensor installed on the eating utensil, the head, or the upper arm. This makes it possible to acquire chewing/swallowing information in everyday life of the user without causing too much stress to the user.

The sensing unit 204 is implemented as, for example, a distance sensor. In this case, the distance sensor is installed on a wearable device that measures how much movement occurs in a direction perpendicular to the surface of the skin in association with the user's chewing motion. Exemplary wearable devices include smart eyeglasses, ear-worn earphones or sensor devices, necklaces or necklace speakers to be worn around the neck, and tooth-embedded sensor devices. Chewing of food is accompanied by repeated up and down movements in a direction perpendicular to the surface of the skin. For example, such movements occur in areas such as the lower portion of the jaw, behind the ears, and the temples. User's chewing/swallowing information can be thus acquired by measuring how much movement occurs in a direction perpendicular to the surface of the skin in each of these areas. This makes it possible to acquire chewing/swallowing information in everyday life of the user without causing too much stress to the user.

The sensing unit 204 may be implemented as an electromyographic sensor that detects electromyographic potentials. When the user chews a food, the electromyographic potentials of muscles around the jaw joint change. Accordingly, the embodiment may use, as chewing/swallowing information, electromyographic information representing the electromyographic potentials of the muscles around the jaw joint that have been detected by the electromyographic sensor. In this case, the electromyographic sensor may be installed on the earpiece of eyeglasses to be worn by the user, or may be installed on an audio output device (e.g., an earphone) to be worn on the ear. This makes it possible to acquire chewing/swallowing information in everyday life of the user without causing too much stress to the user.

The sensing unit 204 may be implemented as a microphone. When the user chews or swallows food, chewing sound or swallowing sound is produced. Accordingly, the embodiment may use, as chewing/swallowing information, sound information representing sound detected by the microphone. In this case, for example, the microphone may be installed on a necklace to be worn by the user, may be installed on an audio output device (e.g., an earphone) to be worn on the ear, or may be embedded in a tooth. If the microphone is installed on a necklace, an audio output device (e.g., an earphone), or a tooth, the installed microphone is located in proximity to the user's mouth, which allows for accurate detection of the chewing sound and swallowing sound. This makes it possible to acquire chewing/swallowing information in everyday life of the user without causing too much stress to the user.

The sensor 200 may, for example, detect sensing data at predetermined sampling intervals, and transmit the detected sensing data at predetermined sampling intervals to the server 300 via the information terminal 100. This allows the server 300 to acquire sensing data in real time.

In the sensor 200, sensing data detected by the sensing unit 204 may be subjected to predetermined computation by the processor 202 to provide chewing/swallowing information with reduced data size. Such chewing/swallowing information may be stored into the memory 203, or analyzed chewing/swallowing information may be transmitted to the information terminal 100 or the food printer 400 via the proximity communications unit 201.

The server 300 includes a communications unit 301, a processor 302, and a memory 303. The communications unit 301 is implemented as a communications circuit for connecting the server 300 to the network 500. The communications unit 301 receives sensing data detected by the sensor 200 and transmitted by the information terminal 100. The communications unit 301 transmits print control information generated by the processor 302 to the food printer 400.

The processor 302 is implemented as, for example, a CPU. The processor 302 acquires chewing/swallowing information from the sensor 200 via the network 500, the chewing/swallowing information being information related to chewing of the user when the user eats a first printed food. More specifically, the processor 302 acquires chewing/swallowing information from sensing data received by the communications unit 301. The first printed food is a food created by the food printer 400 by using a material in paste form and by using a first print pattern.

The processor 302 determines, based on the acquired chewing/swallowing information, the number of chews made by the user, and determines, based on the first print pattern and the number of chews, a second print pattern for a second printed food to be created by the food printer 400. The processor 302 generates print control information for causing the food printer 400 to create the second printed food. The processor 302 transmits the generated print control information to the food printer 400 via the communications unit 301. The print control information includes information such as three-dimensional geometry data, and paste material information. The three-dimensional geometry data represents the geometry of a printed food at the time of printing (prior to heating or other cooking process). The paste material information represents the following pieces of information associated with the three-dimensional geometry data: identification information of a paste material to be printed; and information about where the paste material is to be used. That is, the three-dimensional geometry data may include information such as, for example, what kind of paste is to be used where on the printed food.

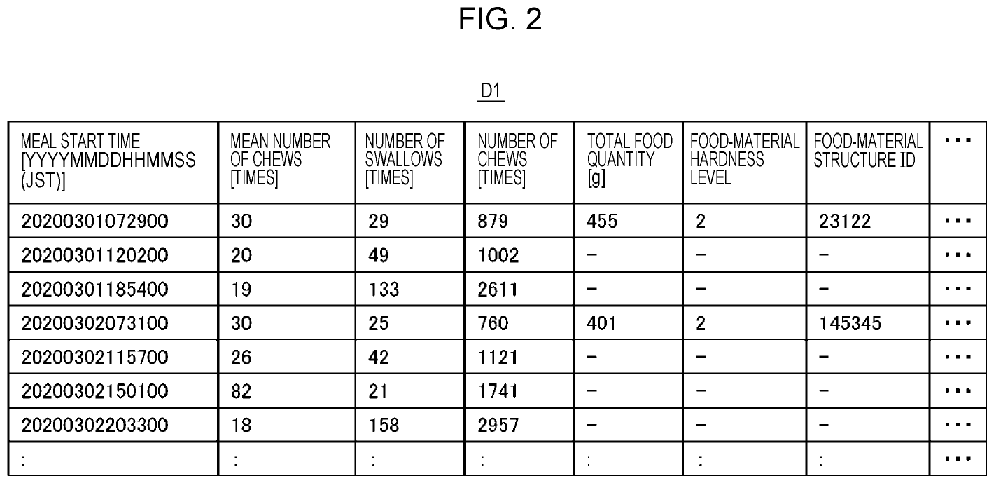

The memory 303 is implemented as a mass storage device such as a hard disk drive or a solid-state drive. The memory 303 stores information such as a chewing database that manages user's chewing/swallowing information. FIG. 2 illustrates an exemplary data structure of a chewing/swallowing information database D1.

A single record in the chewing/swallowing information database D1 stores chewing/swallowing information associated with a single meal. A single meal corresponds to, for example, a meal such as breakfast, lunch, dinner, or a snack. The chewing/swallowing information database D1 stores, with respect to a given single user, chewing/swallowing information for each of meals such as breakfast and lunch. The example in FIG. 2 provides that the user is to eat only a printed food created by a food printer for every breakfast. Symbols “-” in the chewing/swallowing information database D1 indicate that the corresponding pieces of information have not been successfully obtained.

The chewing/swallowing information database D1 stores the following and other pieces of information in association with each other: meal start time, the mean number of chews, the number of swallows, the number of chews, total food quantity, food-material hardness level, and food-material structure ID. Meal start time represents the start time of a single meal. For example, for a case where the sensor 200 is implemented as an acceleration sensor, if the acceleration sensor of the processor 302 detects an acceleration waveform representative of raising or lowering of an eating utensil after such acceleration waveform has not been detected for a certain period of time, the time at which the waveform is detected is identified as the meal start time. Alternatively, the user may input a command to the information terminal 100 that signals the start of a meal, and the time at which the server 300 receives the command may be used to represent the meal start time.

The mean number of chews is calculated as the number of chews divided by the number of swallows within a single meal. The number of swallows represents the number of times the user has swallowed food during a single meal. To determine the number of swallows, the processor 302 may analyze chewing/swallowing information acquired from the sensor 200 to identify each individual swallowing motion, and count how many times such a swallowing motion has been repeated. The mean number of chews corresponds to the mean of the numbers of chews taken until a single swallow occurs.

Swallow cycle duration represents the period of time from when the user starts chewing a bite of food to when the user swallows the bite of food. For example, if the sensor 200 is implemented as an acceleration sensor, the processor 302 may analyze acceleration information acquired from the acceleration sensor, and detect the timing of raising of an eating utensil (first timing) or the timing of lowering of an eating utensil (second timing) to thereby identify the beginning of the current swallow cycle duration. The processor 302 may then determine the time interval between the beginning of the current swallow cycle duration and the beginning of the next swallow cycle duration as representing one swallow cycle duration. Chewing is sometimes paused after a bite of food is swallowed. After a meal is finished, chewing does not occur until the next meal is started. Accordingly, if detection of the beginning of the current swallow cycle duration is not followed by detection of the beginning of the next swallow cycle duration for a predetermined period of time or more, the processor 302 may regard the moment of elapse of the predetermined period of time as representing the end of the current swallow cycle duration, and thus identify each swallow cycle duration. Alternatively, the processor 302 may regard the timing at which an eating utensil is lowered and stops moving as representing the end of the current swallow cycle duration, and thus identify each swallow cycle duration. The timing of raising or lowering of an eating utensil can be detected through, for example, pattern matching between a predefined acceleration waveform representative of raising of the eating utensil or a predefined acceleration waveform representative of lowering of the eating utensil, and acceleration information acquired from the acceleration sensor.

If the sensor 200 is implemented as an electromyographic sensor, the processor 302 may, for example, analyze electromyographic information acquired from the electromyographic sensor to detect the start timing and end timing of chewing for a bite of food, and determine the time interval between the start timing and the end timing as the swallow cycle duration. It is presumed that for a bite of food, the electromyographic potential changes in a specific pattern during the period of time from the start of chewing to the moment of swallowing. Accordingly, the processor 302 may detect, from electromyographic information, the timing of chewing initiation and the timing of swallowing with respect to a bite of food by using pattern matching or other methods, and detect the period of time between these two timings as the swallow cycle duration.

If the sensor 200 is implemented as a microphone, the processor 302 may, for example, analyze sound information acquired from the microphone to detect the timing of occurrence of chewing sound, which represents the timing of chewing initiation with respect to a bite of food, and the timing of swallowing, which represents the timing when the bite of food is swallowed, and the processor 302 may then determine the time interval between these two timings as the swallow cycle duration. For a bite of food, chewing sound is produced when chewing is initiated, and swallowing sound is produced at the timing of swallowing. Accordingly, the processor 302 may detect such chewing sound and swallowing sound from sound information by using pattern matching or other methods.

The number of chews is defined as the number of times the user has chewed food during a single meal. The number of chews is in a proportional relationship with the time taken to eat the meal (mean duration). If the sensor 200 is implemented as an acceleration sensor, the processor 302 may determine the meal duration as the amount of time during which chews occurring consecutively within a predetermined time interval of each other continue, and measure the number of chews made within the determined meal duration.

If the sensor 200 is implemented as a distance sensor, the processor 302 counts the number of occurrences of a distance pattern representing a single chew and/or swallow, from distance information representative of the up and down movements that occur in a direction perpendicular to the surface of the skin in association with each chew and/or swallow during a single meal. The sensor 200 thus calculates the meal duration and the number of chews in a manner similar to that mentioned above. Meal duration is defined as the amount of time during which distance patterns representing chewing and/or swallowing and occurring consecutively within a predetermined time interval of each other are counted continuously. The number of chews can be determined by counting the number of occurrences of distance patterns representative of chewing within the meal duration.

If the sensor 200 is implemented as an electromyographic sensor, the processor 302 analyzes electromyographic information representing individual chews and/or swallows during a single meal, and counts the number of occurrences of electromyographic patterns each representing a single chew and/or swallow. The processor 302 thus calculates the meal duration and the number of chews in a manner similar to that mentioned above. Meal duration is defined as the amount of time during which electromyographic patterns representing chewing and/or swallowing and occurring consecutively within a predetermined time interval of each other are counted continuously. The number of chews can be determined by counting the number of occurrences of electromyographic patterns representative of chewing within the meal duration.

If the sensor 200 is implemented as a microphone, the processor 302 counts, from sound information representing individual chews and/or swallows during a single meal, the number of occurrences of sound patterns each representing a single chewing sound and/or swallowing sound. The processor 302 thus calculates meal duration and the number of chews. Meal duration is defined as the amount of time during which chewing sounds and/or swallowing sounds occurring consecutively within a predetermined time interval of each other are counted continuously. The number of chews can be determined by counting the number of occurrences of sound patterns each representing a chewing sound within the meal duration.

Total food quantity is defined as the total weight of food taken by the user in a single meal. The present example provides that the user is to eat a printed food for every breakfast. Since it is the server 300 that instructs that the printed food be created, the server 300 is able to determine the weight of the printed food that the user eats for every breakfast, from the weight of a paste used for creating the printed food. Accordingly, for breakfast, the processor 302 may calculate the total weight from the weight of a paste that the processor 302 has specified when generating print control information. In this regard, whether a given piece of chewing/swallowing information pertains to breakfast can be determined from the meal start time corresponding to the piece of chewing/swallowing information.

In the example in FIG. 2, the total food quantity has not been successfully identified for meals other than breakfast, and thus the Total Food Quantity cells corresponding to the chewing/swallowing information for meals other than breakfast are marked “-”. It is to be noted, however, that if the total food quantity has been successfully detected for a meal other than breakfast, the detected total food quantity is written into the chewing/swallowing information database D1. For example, when taking a meal, the user is made to capture an image of the prepared meal with a camera and have the captured image transmitted to the server 300. The processor 302 may then analyze the captured image of the prepared meal to determine the total food quantity. Alternatively, if a weight sensor is installed on the eating utensil being used, the processor 302 may determine the total food quantity by adding up the weight of each bite of food detected by the weight sensor over the entire duration of a single meal.

Food-material hardness level is a numerical value representing a graded measure of the chewing force (biting force) and swallowing force required for eating a food material. As for the food-material hardness level, for example, the classification for different classes of food materials described at the website “https://www.udfjp/about_udf/section_01.html” may be used. The lower the hardness level of a food material, the harder the food material. In the example in FIG. 2, the food-material hardness level has not been successfully identified for meals other than breakfast, which is a meal for which only a printed food is to be eaten, and thus the Food-Material Hardness Level cells corresponding to the chewing/swallowing information for meals other than breakfast are marked “-”. It is to be noted, however, that if the food-material hardness level has been successfully identified through analysis of an image of a prepared meal, the identified food-material hardness level is written into the chewing/swallowing information database D1.

The food-material hardness level of a food as a measure of its chewiness may represent how much in terms of volume or volume proportion the food includes a portion with a predetermined food-material hardness. For example, it is known that the presence of root vegetables cut into large pieces in a food contributes to chewiness and leads to increased number of chews. By contrast, the presence of root vegetables cut into small pieces does not contribute very much to chewiness and leads to reduced number of chews. As described above, the expected number of chews for a food varies with its internal structure, that is, how much in terms of volume or volume proportion the food includes a portion with a hardness greater than or equal to a certain value that provides a chewy sensation. Mass may be used instead of volume, in which case the food-material hardness level of a food may be a measure representing how much in terms of mass or mass proportion the food includes a portion with a predetermined food-material hardness.

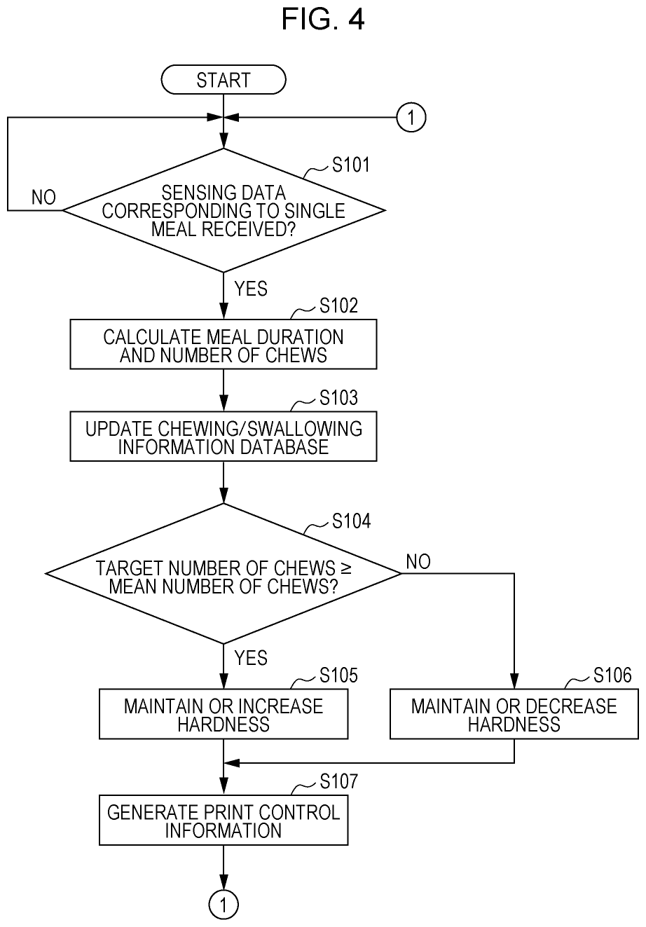

As for the food-material hardness level, the processor 302 may determine which one of the above-mentioned classes a hardness set at step S105 or step S106 described later with reference to FIG. 4 corresponds to, and write the determined class into the corresponding Food-Material Hardness Level cell.

Food-material structure ID is an identifier of the three-dimensional geometry data of a printed food created by the food printer 400. The three-dimensional geometry data is, for example, CAD data. In the example in FIG. 2, the food-material structure ID is written only for the chewing/swallowing information corresponding to breakfast for which the printed food is eaten.

In the example in FIG. 2, the chewing/swallowing information database D1 stores chewing/swallowing information for each single meal. However, this is not intended to limit the present disclosure. For example, the chewing/swallowing information database D1 may store chewing/swallowing information for each single swallow. Alternatively, the chewing/swallowing information database D1 may store chewing/swallowing information every time a bite of food is swallowed. Although the chewing/swallowing information database D1 in FIG. 2 stores chewing/swallowing information for a given single user, the chewing/swallowing information database D1 may store chewing/swallowing information for plural users. In this case, providing the chewing/swallowing information database D1 with a user ID field makes it possible to identify which piece of chewing/swallowing information corresponds to which user.

Although the foregoing description assumes that total food quantity, food-material hardness level, and food-material structure ID are known only for printed foods created by the food printer 400, this is not intended to be limiting. Generally, for any food sold as a finished product, such as bread, confectionery, packed meal, canned food, instant food, or boil-in-the-bag food, the total weight, hardness level, structure, and other information about such a food are known. Accordingly, if it can be determined from sensing data obtained from a camera or other devices that a food eaten by the user corresponds to such a food, values corresponding to the food may be registered into the chewing/swallowing information database D1. This configuration is useful for the ability to accurately acquire user's chewing/swallowing information, chewing ability, and/or swallowing ability also from foods other than those created by the food printer 400.

Reference now returns to FIG. 1. The food printer 400 is a food preparation apparatus that shapes a food by dispensing a gelled food material (paste) and depositing the dispensed food material in layers.

The food printer 400 includes a communications unit 401, a memory 402, a paste dispenser 403, a controller 404, a UI unit 405, and a laser output unit 406. The communications unit 401 is implemented as a communications circuit for connecting the food printer 400 to the network 500. The communications unit 401 receives print control information from the server 300. The memory 402 is implemented as a rewritable non-volatile storage device such as a flash memory. The memory 402 stores print control information transmitted from the server 300.

The paste dispenser 403 includes plural slots, and a nozzle for dispensing a paste loaded in each slot. Each slot can be loaded with a different type of paste. Each paste is a food material packaged according to its type. The paste to be used can be replaced with respect to the paste dispenser 403. The paste dispenser 403 repeats a process of dispensing a paste while moving the nozzle in accordance with print control information. The paste is thus deposited in sequential layers to thereby shape a printed food.

The laser output unit 406 applies, in accordance with print control information, a laser beam or infrared radiation to the paste dispensed by the paste dispenser 403. The laser output unit 406 thus heats a portion of the paste to brown a printed food or shape a printed food. The laser output unit 406 is also capable of adjusting the power of the laser beam or infrared radiation to adjust the temperature at which to bake a printed food to thereby adjust the hardness of the printed food. The food printer 400 is capable of causing the paste dispenser 403 to discharge a paste while causing the laser output unit 406 to apply a laser beam. This makes it possible to simultaneously perform shaping and thermal cooking of the printed food.

The food printer 400 may include a temperature sensor (not illustrated) for measuring the temperature of a printed food that has been irradiated by the laser output unit 406. The food printer 400 may thus detect the heating condition for each individual portion of the printed food, and allow each individual portion of the printed food to be precisely heated to a preset temperature and for a preset amount of time for cooking. Such precise heating makes it possible to achieve a preset food hardness level, texture, or taste.

A setting as to which slot of the paste dispenser 403 is loaded with which paste can be made by using a smartphone application installed on the information terminal 100 that communicates with the food printer. Alternatively, this setting can be made by reading, with a reader attached to each slot, a paste ID stored in an electric circuit attached to the package of a paste, and outputting the read paste ID to the controller 404 in association with the corresponding slot number.

The UI unit 405 is implemented as, for example, a touchscreen display. The UI unit 405 receives an input of a user's instruction, or displays various screens.

The controller 404 is implemented as a CPU or a dedicated electric circuit. The controller 404 creates a printed food by controlling the paste dispenser 403 and the laser output unit 406 in accordance with print control information transmitted from the server 300.

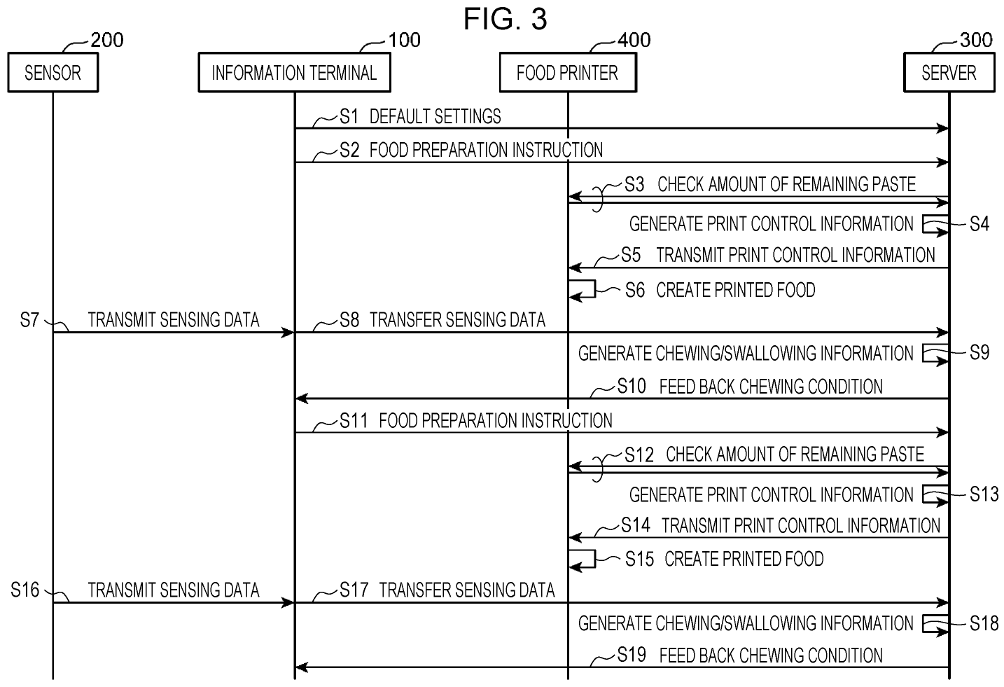

Reference is now made to processing according to the embodiment. FIG. 3 is a sequence diagram illustrating an overview of processing performed by the information system illustrated in FIG. 1.

At step S1, the information terminal 100 receives a user's input related to default settings information required for the user to receive a service from the server 300, and transmits the default settings information to the server 300. The default settings information includes, for example, a target number of chews (an example of a predetermined number of chews), which is a target number of chews for chewing a bite of food. The target number of chews is, for example, is about 30. The default settings information may include the setting of a target number of chews that the user wishes to achieve for a single meal and/or for a single day's meals.

Subsequently, at step S2, the information terminal 100 receives a user's input of a food preparation instruction, which is an instruction for causing the food printer 400 to start preparation of a printed food, and transmits the instruction to the server 300.

Subsequently, at step S3, the server 300 transmits a check signal for causing the food printer 400 to check the amount of remaining paste, and receives a response from the food printer 400. In response to receiving the check signal, the food printer 400 detects, for example, the amount of paste remaining in the paste dispenser 403. If the amount of remaining paste is greater than or equal to a predetermined value, the food printer 400 transmits a response to the server 300 that indicates that creation of the printed food is possible. If the amount of remaining paste is less than the predetermined value, the food printer 400 transmits a response to the server 300 that indicates that creation of the printed food is not possible. In this case, the server 300 may transmit a message to the information terminal 100 that prompts the user to load more paste, and wait on standby until the server 300 receives a response indicating that creation of the printed food is possible.

Subsequently, at step S4, the server 300 generates print control information. Further details about the generation of print control information will be given later with reference to FIG. 4.

At step S5, the server 300 transmits the print control information to the food printer 400. Since no sensing data for a user who has eaten the printed food has been obtained at this point, the server 300 generates the print control information based on, for example, the default hardness of the printed food. The default hardness corresponds to an example of the first print pattern.

At step S6, the food printer 400 creates the printed food in accordance with the received print control information. The printed food created at this time corresponds to an example of the first printed food. At step S7, the sensor 200 transmits sensing data to the information terminal 100. The sensing data includes the chewing/swallowing information of the user who has eaten the printed food created at step S6. At step S8, the information terminal 100 transfers the sensing data transmitted at step S7 to the server 300.

In this regard, the sensing data may be primary data (data including the raw data from the sensing unit 204) representing the user's chewing/swallowing information detected by the sensor 200, or may be secondary data (smaller-sized processed data obtained through computational processing of the primary data and related to the user's chewing/swallowing information, e.g., information about the number of chews and the meal duration) that has been computationally processed by the processor 202.

At step S9, the server 300 generates chewing/swallowing information associated with a single meal based on the sensing data transmitted to the server 300, and updates the chewing/swallowing information database D1 by using the chewing/swallowing information.

At step S10, the server 300 generates chewing condition data based on the chewing/swallowing information generated at step S9, and transmits the chewing condition data to the information terminal 100 to provide feedback of the chewing condition to the user. The chewing condition data includes, for example, the information illustrated in FIG. 2, such as the mean number of chews, the number of swallows, the number of chews, total food quantity, and food-material hardness level. The chewing condition data is displayed on the display 106 of the information terminal 100.

At step S11, the information terminal 100 transmits the food preparation instruction described above with reference to step S2 to the server 300. At step S12, the server 300 checks the amount of paste remaining in the food printer 400 in the same manner as step S3.

At step S13, the server 300 compares the number of chews included in the chewing/swallowing information generated at step S9 with a target number of chews, and based on the comparison result, the server 300 determines a print pattern (including not only three-dimensional geometry data representing a three-dimensional geometry in which to deposit a paste material in layers but also the identification information of the paste material to be used) for a printed food and, as required, a thermal cooking method, and generates print control information based on the determined print pattern (and, as required, the determined thermal cooking method). Further details about this process will be given later with reference to the flowchart of FIG. 4. The hardness determined at this time corresponds to an example of the second print pattern. The printed food created in accordance with the print control information generated at this time corresponds to an example of the second printed food.

Steps S14, S15, S16, S17, S18, and S19 are similar to steps S5, S6, S7, S8, S9, and S10. Thereafter, the processing from steps S11 to S19 is repeated, and the chewing and swallowing function of the user is gradually improved.

FIG. 4 is a flowchart according to the embodiment, providing a detailed illustration of processing performed by the server 300. The processor 302 of the server 300 determines whether sensing data corresponding to a single meal for a printed food has been received by the communications unit 301 (step S101). For example, as for the start timing of a single meal (meal start time), when a change is observed in the sensing data provided from the sensor 200 after no change in the sensing data has been observed for a predetermined amount of time or more, the timing of the observed change corresponds to the start timing. As for the end timing of a single meal (meal end time), for example, when a predetermined amount of time or more elapses after a change in the sensing data ceases to be observed, the timing at which a change in the sensing data ceases to be observed corresponds to the end timing of a single meal. In the example in FIG. 2, a printed food is eaten for every breakfast. Accordingly, if the start timing of a meal falls within the time of day for breakfast, the processor 302 may determine that the sensing data corresponding to a single meal acquired at step S101 represents sensing data for the printed food.

Alternatively, the sensing data corresponding to a single meal acquired most recently after transmission of print control information may be determined as sensing data for the printed food. Alternatively, if an indication of the start of a meal and an indication of the end of the meal have been input by the user to the information terminal 100, a series of sensing data acquired in this case may be determined to be sensing data corresponding to a single meal.

At step S102, the processor 302 calculates the meal duration and the number of chews from the sensing data corresponding to a single meal. Since the details of how to calculate the meal duration and the number of chews have been described above, no further description in this regard will be provided herein. At step S102, in addition to calculation of the number of chews, values such as the number of swallows, the mean number of chews, and the total food quantity are also calculated, and the chewing/swallowing information illustrated in FIG. 2 is generated based on the results of these calculations.

At step S103, the processor 302 updates the chewing/swallowing information database D1 by using the chewing/swallowing information calculated at step S102.

At step S104, the processor 302 determines whether a target number of chews per swallow is greater than or equal to the mean number of chews. If the target number of chews is greater than or equal to the mean number of chews (YES at S104), the processor 302 controls the print pattern so as to increase the number of chews such as by maintaining or increasing the hardness of the printed food relative to the previous value. The previous value refers to the value of the hardness of the printed food last eaten by the user. The hardness represented by the previous value corresponds to an example of the first print pattern. In increasing the hardness of the printed food, the processor 302 may add a predefined amount of change of hardness to the previous value to thereby increase the hardness.

If the target number of chews is less than the mean number of chews (NO at S104), the processor 302 controls the print pattern to adjust the number of chews, such as by maintaining or decreasing the hardness of the printed food relative to the previous value (step S106). In decreasing the hardness of the printed food, the processor 302 may subtract the amount of change mentioned above from the previous value to thereby decrease the hardness. Exemplary conceivable cases where the hardness is maintained include when the number of times that the printed food of the same hardness has been given to the user is less than a predetermined number of times.

At step S107, based on the hardness that has been maintained increased, or decreased, the processor 302 generates print control information including a print pattern, and returns the processing to step S101.

As the above-mentioned processing is repeated, for a user for whom the target number of chews per swallow is greater than or equal to the mean number of chews, the hardness of the printed food is maintained or gradually increased. Accordingly, a user with decreased chewing and swallowing function is given a somewhat soft printed food at first, and then sequentially given printed foods with gradually increased hardness. This helps to efficiently improve the chewing and swallowing function of such a user.

As for a user with the target number of chews less than the mean number of chews, the hardness of the printed food is maintained or gradually decreased. Therefore, for a user with an excessively large number of chews, the number of chews is allowed to progressively converge to an appropriate value.

The foregoing description is directed to a case where, with respect to the number of chews per swallow, a target value is compared with a mean value obtained by actual measurement from sensing data. However, this is not intended to limit the present disclosure. For example, with respect to the number of chews per meal, a target value may be compared with an actual measurement actually measured from sensing data to thereby similarly generate print control information including a print pattern and a thermal cooking method for a second printed food. If print control information is generated by focusing only on an increase or decrease in the number of chews per swallow, this may cause the number of chews for a single whole meal to decrease. Therefore, print control information may be generated based on the number of chews and/or the meal duration for a single meal. Exemplary conceivable cases where the number of chews during the whole meal decreases even though the number of chews per swallow increases include when a large amount of food is eaten per bite.

Detailed reference is now made to generation of print control information. According to the embodiment, the hardness of a printed food is adjusted by using one of the approaches described below. Accordingly, the print control information to be generated differs depending on which variation is used.

In the first variation, a printed food is formed as a three-dimensional structure with plural holes, and the number of these holes is increased or decreased to adjust the hardness of the printed food. A printed food becomes softer as the number of holes in the printed food increases, and harder as the number of holes decreases. Accordingly, in the first variation, the hardness of a printed food is adjusted by specifying the number of holes per unit volume of the printed food. Such adjustment of the number of holes can be made by changing three-dimensional geometry data. In this sense, it can be conversely stated that such holes represent a print pattern for forming a printed food. This is because such a print pattern causes the printed food to have portions with no paste material deposited in layers (i.e., holes).

Once the processor 302 of the server 300 determines the hardness of the printed food at step S105 or step S106, the processor 302 determines the number of holes per unit volume or print pattern that is previously defined for achieving the hardness. The processor 302 then extracts or generates a print pattern (to be referred to also as “three-dimensional geometry data”) for creating a printed food that has a specified number of holes per unit volume or has a specified three-dimensional structure.

For example, the processor 302 may correct the default three-dimensional geometry data such that the number of holes per unit volume in the default three-dimensional geometry data becomes equal to the specified number of holes per unit volume. All holes may or may not have the same diameter. One non-limiting example of the basic geometry of the default three-dimensional geometry data is a cuboid. Three-dimensional geometry data generated by the processor 302 already reflects a hardness as determined by the number of holes per unit volume. Therefore, according to the first variation, print control information may include three-dimensional geometry data generated by the processor 302, and may not include hardness data.

Print control information may include, as a print pattern (three-dimensional geometry data), identification information representing what kind of paste material is to be used for each print location. This allows the internal structure of a printed food to include portions that differ in color, hardness, and/or taste.

However, this is intended to be illustratively only. Alternatively, for example, the controller 404 of the food printer 400 may correct the default three-dimensional geometry data from hardness data. In this case, hardness data and the default three-dimensional geometry data may be included in print control information.

In the second variation, a printed food is formed as a three-dimensional structure with plural layers, and the individual layers are varied in hardness, paste material, and/or print pattern (three-dimensional geometry data) to thereby increase or decrease the number of chews or meal duration for the printed food. For example, a food with a hard surface and a soft interior such as rice cracker can give the user a texture sensation such that as the user crushes its hard surface with the teeth, its contents with taste mix with saliva and melt out from the inside. This induces saliva production, which helps to efficiently improve the chewing and swallowing function of the user. Accordingly, in the second variation, for example, the printed food includes a first layer having a third hardness, and a second layer having a fourth hardness lower than the third hardness. The printed food is created by stacking the first layer, the second layer, and the first layer in this order. This allows the resulting printed food to have an internal structure with a three-dimensional arrangement of chewy and non-chewy portions. In addition to providing the printed food with a three-dimensional, rather than monotonous, texture that the user does not get tired of, such an internal structure is also expected to result in increased number of chews as the user thoroughly crushes the hard portions with the teeth before swallowing, which can potentially lead to improved chewing and swallowing function of the user.

In this case, the processor 302 of the server 300 determines, with respect to the hardness or the number of chews set at step S105 or step S106, a predefined hardness or a predefined number of chews as a value representative of a third hardness, a third paste material, or a third print pattern, and of a fourth hardness, a fourth paste material, or a fourth print pattern. The processor 302 may then generate print control information including the following pieces of information: three-dimensional geometry data representing a print pattern including the specification of which paste material is to be used; the third hardness; and the fourth hardness. In this case, the three-dimensional geometry data may include data indicating which region corresponds to the first layer and which region corresponds to the second layer. In the second variation mentioned above, the hardness adjustment for the first and second layers may be made based on the number of holes or print pattern described above with reference to the first variation. In another example, the hardness adjustment may be made by varying the type of paste. In this case, print control information may include information that specifies the type of paste used for the first layer and the type of paste used for the second layer. In another example, the hardness adjustment may be made by varying the print pattern (three-dimensional geometry data for the paste material). In this case, print control information may include information that specifies the type of paste used for the first layer and the type of paste used for the second layer.

Although a printed food has been described above as being made up of a second layer sandwiched by two first layers, a printed food may be made up of a first layer and a second layer. Further, if a printed food is made up of a second layer sandwiched by two first layers, the printed food may have a structure such that the first layer includes plural sub-layers of differing hardness, and that the second layer includes plural sub-layers of differing hardness, with the hardness of the resulting printed food decreasing gradually with increasing distance from the surface toward the center.

In the third variation, the hardness of a printed food is adjusted by specifying the temperature (thermal cooking method) used to bake the printed food. The temperature at which to bake a printed food is adjusted by adjusting the power of the laser beam or infrared radiation to be applied. The hardness of a printed food can be changed by adjusting this temperature. In this case, the processor 302 may determine a temperature previously defined for achieving the hardness set at step S105 or S106, and incorporate, into print control information, the determined temperature, and further, as required, information related to a thermal cooking method and representing the amount of time for which to maintain the temperature. In this case, the print control information may include the following pieces of information: three-dimensional geometry data serving as a print pattern; information representing the type of paste to be used; the heating temperature associated with the three-dimensional geometry data; and further, as required, the heating time at the heating temperature.

Various parameters included in print control information correspond to an example of a printing condition for, if the number of chews made by the user is less than a predetermined number of chews, creating a second printed food according to a second print pattern that is used to increase the number of chews.

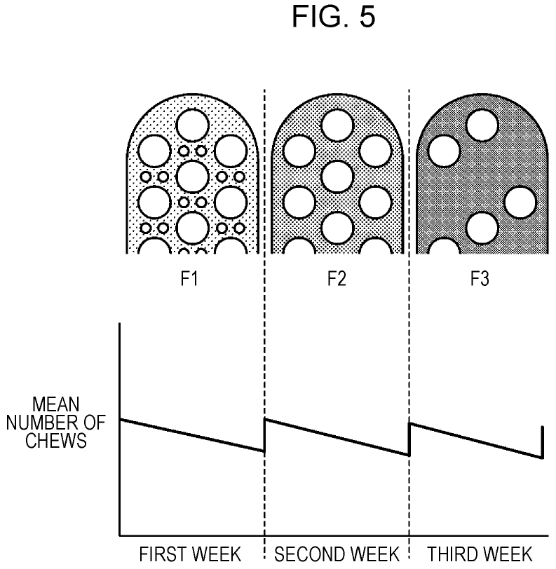

FIG. 5 illustrates the progression of the mean number of chews over time. In this example, the flowchart in FIG. 4 is conducted on a weekly basis, and a printed food of the same hardness is provided to the user every morning for each week. In the first week, the user eats a printed food of a hardness F1 every morning. As the user thus gets used to the printed food of the hardness F1, the chewing and swallowing function of the user gradually improves, and the mean number of chews preceding a single swallow gradually decreases.

At the beginning of the second week, it is determined whether the mean number of chews is greater than or equal to a target number of chews. At this point, the mean number of chews is not greater than the target number of chews. Accordingly, the user is given a printed food every morning that has a hardness F2, which is a hardness increased from the hardness F1 by a predetermined amount of change. Although this causes the user to increase the mean number of chews for a while to crush the printed food of the hardness F2 with the teeth, the chewing and swallowing function of the user then gradually improves, which leads to progressively decreasing mean number of chews. Likewise, in the third week, the user is given a printed food every morning that has a hardness F3, which is a hardness increased from the hardness F2 by a predetermined amount of change. Although this causes the user to increase the mean number of chews for a while to crush the printed food of the hardness F3 with the teeth, the chewing and swallowing function of the user then gradually improves, which leads to progressively decreasing mean number of chews. Thereafter, until the mean number of chews exceeds the target number of chews, the hardness of the printed food given to the user is gradually increased, which allows the chewing and swallowing function of the user to improve progressively.

Although the foregoing description is directed to updating print control information by using the mean number of chews preceding a single swallow, this is not intended to limit the present disclosure. Instead of the mean number of chews preceding a single swallow, the number of chews required for a single whole meal may be used.

The present disclosure may take various modifications as given below.

(1) Although FIG. 1 depicts an example in which the sensor 200 transmits sensing data to the server 300 via the information terminal 100, alternatively, the sensor 200 may be connected to the network 500. In this case, sensing data may be transmitted from the sensor 200 to the server 300 or the food printer 400 without passing through the information terminal 100.

(2) The sensor 200 may be implemented as a camera. In this case, the sensor 200 is placed in a room where the user takes a meal. Generally speaking, cameras (edge terminals) have advanced processing capabilities. This means that by analyzing an image captured with such a camera, the mean number of chews can be calculated or inferred by using a neural network model. Accordingly, in this modification, the processor 202 of the sensor 200 calculates the mean number of chews by analyzing an image captured by the sensing unit 204. Chewing/swallowing information representing the calculated mean number of chews is then incorporated into sensing data, and transmitted to the server 300 or the food printer 400.

In this case, the mean number of chews is included in the chewing/swallowing information. The server 300 is thus able to determine whether the mean number of chews is greater than or equal to a target number of chews, without calculating the mean number of chews. This allows for reduced processing load on the server 300.

If a camera is used to measure chewing and swallowing through analysis, by also analyzing the lateral movements of the upper and lower jaws to measure the number of times food is chewed with the right teeth, and the number of times food is chewed with the left teeth, the user's uneven chewing can be also measured. If the difference in the number of chews between the right and left sides is greater than a predetermined value (i.e., if uneven chewing is suspected), the server 300 may register the number of chews on the left side and the number of chews on the right side into the chewing/swallowing information database D1 individually. Notification of information indicative of such uneven chewing may be provided to the user via the information terminal 100 at step S10 or S19 to allow the user to have the consciousness or motivation to improve uneven chewing (i.e., make the number of chews more even between the left and right sides). For example, the chewing balance between the right and left sides may be presented in quantified or visualized form. It is difficult for the user to notice uneven chewing on his or her own, which occurs as the jaws or masticatory muscles on the habitual chewing side become strained while the masticatory muscles on the other side relax and which can lead to misaligned jaws and consequently misalignment or distortion of the entire body. Such uneven chewing can be expected to be prevented or improved by measuring the uneven chewing with the sensor 200, and providing appropriate feedback to the user via the information terminal 100 as described above.

If a camera is to be used to measure chewing and swallowing, it is also possible to use the information terminal 100 with a built-in camera, such as a smartphone, as the sensor 200. As described above, by detecting the chewing and swallowing behavior of the user during meal intake via the camera, and applying an image recognition process to the detection results, values such as the mean number of chews made by the user prior to a single swallow for a food included in the meal, and the number of chews and meal duration taken for the single whole meal can be measured. In another conceivable example, a camera installed on a device (robot) capable of autonomous movement may be used in the manner as described above. In this case, the autonomous device (robot) is capable of, in response to detecting that the user has started taking a meal, detecting the user's chewing and swallowing behavior during the meal as described above with the installed camera, and acquiring associated chewing/swallowing information while the user takes the meal. Using the autonomous device (robot) as described above allows for continuous recording of chewing/swallowing information without the trouble of requiring the user to make manual settings while taking the meal.

The condition of uneven chewing mentioned above may be measured not by using a camera but by measuring the electromyographic potential or momentum of each of the left and right masticatory muscles of the user's face. During chewing, the masticatory muscle (at least one of the masseter muscle, the temporalis muscle, the lateral pterygoid muscle, or the medial pterygoid muscle) on either the right side or left side on which the user tends to chew habitually is used more than that on the other side. Accordingly, the condition of the user's uneven chewing can be measured also by measuring the electromyographic potential or momentum of each of the left and right masticatory muscles.

According to this modification, the processor 202 may, for example, apply a predetermined image recognition process for detecting whether the user is chewing to an image captured by the sensing unit 204. In this way, the processor 202 may detect values such as the number of swallows and the number of chews within a single meal, and calculate the mean number of chews. For example, the processor 202 may detect features of the user's mouth, and keep track of the features. If the behaviors of the tracked features are representative of repeated opening and closing movements of the upper and lower jaws, the processor 202 may determine that the user is making chewing motion. The processor 202 may calculate the number of swallows and the number of chews from the detection results, and calculate the mean number of chews or other values from these calculated values.

According to this modification, the sensing unit 204 is capable of capturing an image of a prepared meal. The processor 202 is thus able to calculate the total food quantity by analyzing the image of the prepared meal. According to this modification, the processor 202 may incorporate, in addition to the mean number of chews, the following pieces of information associated with a single meal into chewing/swallowing information: the number of swallows, the number of chews, and the total food quantity.

The foregoing description herein is directed to an exemplary case in which, based on information acquired from the information terminal 100, the sensor 200, and the food printer 400, the server 300 executes processing such as generation of print control information (step S4) and generation of chewing/swallowing information (step S9). However, this is not intended to limit the present disclosure. For example, the food printer 400 may execute the above-mentioned processing based on information acquired from the information terminal 100 and the sensor 200. Further, the food printer 400 may directly acquire sensing data (see step S7) from the sensor 200 rather than via the information terminal 100.

Aspects of the present disclosure make it possible to efficiently improve chewing and swallowing function, and therefore find utility in industrial fields aimed at promoting health.

What is claimed is:

1. A method for controlling a food printer in a food-material providing system, the food printer being a food printer that creates a first printed food, the first printed food being created by the food printer by using a first print pattern, the method comprising:
acquiring chewing/swallowing information via a network from a sensing device associated with a user, wherein the chewing/swallowing information is related to chewing of the user when the user eats the first printed food;
determining based on the chewing/swallowing information, a number of chews made by the user in eating the first printed food, and determining based on at least the first print pattern and the number of chews, a second print pattern used for a second printed food to be created by the food printer; and
transmitting print control information to the food printer via the network, wherein the print control information is used for causing the food printer to create the second printed food using the determined second print pattern.

2. A method for controlling a food printer in a food-material providing system, the food printer being a food printer that creates a first printed food, the first printed food being created by the food printer by using a first print pattern, the method comprising:
acquiring chewing/swallowing information via a network from a sensing device associated with a user, wherein the chewing/swallowing information represents a number of chews made by the user in eating the first printed food;
determining based on at least the first print pattern and the number of chews, a second print pattern used for a second printed food to be created by the food printer; and
transmitting print control information to the food printer via the network, wherein the print control information is used for causing the food printer to create the second printed food using the determined second print pattern.

3. The control method according to claim 1, wherein
the number of chews includes a total number of times chewing is made by the user in eating the first printed food.

4. The control method according to claim 1, wherein
the print control information includes a print condition for, if the number of chews made by the user is less than a predetermined number of chews, creating the second printed food that has a smaller mass per unit volume than the first printed food.

5. The control method according to claim 1, wherein
if the first printed food includes a plurality of chunks of food in a bite size of less than or equal to 15 cubic centimeters, and the number of chews made by the user is less than a predetermined number of chews, the print control information includes a print condition for creating the second printed food that includes a plurality of chunks of food in a bite size of less than or equal to 15 cubic centimeters and that has a mean volume that is less than a mean volume of the plurality of chunks of food in the bite size included in the first printed food.

6. The control method according to claim 1, wherein
the print control information includes a print condition for, if the number of chews made by the user is less than a predetermined number of chews, creating the second printed food that has a greater volume of a hard portion than the first printed food, wherein the hard portion has a hardness greater than or equal to a predetermined hardness.

7. The control method according to claim 1, wherein
the sensing device includes an acceleration sensor, and
the chewing/swallowing information includes acceleration information that represents an acceleration detected by the acceleration sensor.

8. The control method according to claim 1, wherein
the sensing device includes a distance sensor, and
the chewing/swallowing information includes distance information that is detected by the distance sensor and that represents a distance to a skin.

9. The control method according to claim 1, wherein
the sensing device detects an electromyographic potential, and
the number of chews is determined based on the detected electromyographic potential.

10. The control method according to claim 1, wherein
the sensing device detects chewing sound, and
the number of chews is determined based on the detected chewing sound.

11. The control method according to claim 1, wherein
the sensing device includes a camera, and
the number of chews made by the user is determined based on a result of image recognition performed by using an image obtained with the camera.

12. The control method according to claim 1, wherein
the sensing device is installed on an autonomous device that performs sensing on the user.

13. The control method according to claim 1, wherein
the sensing device is installed on eyeglasses of the user.

14. The control method according to claim 1, wherein
the sensing device is installed on a device to be worn around a neck of the user.

15. The control method according to claim 1, wherein
the sensing device is installed on a device to be worn on an ear of the user.

16. The control method according to claim 1, wherein
the second printed food is created by using a plurality of paste materials, and
the second print pattern specifies where each of the plurality of paste materials is to be used.

17. The method according to claim 1, wherein
the second printed food comprises a three-dimensional structure including a plurality of layers, the plurality of layers including a first layer and a second layer, and
the print control information includes a print condition for causing a paste material used for the first layer to be varied from a paste material used for the second layer.

18. The method according to claim 1, wherein
the second printed food comprises a three-dimensional structure including a plurality of layers, the plurality of layers including a first layer and a second layer, and
the print control information includes a print condition for causing a third print pattern used for the first layer to be varied from a fourth print pattern used for the second layer.

19. The method according to claim 1, wherein
the print control information specifies a temperature at which to bake the second printed food.
